(19) United States
(12) Patent Application Publication
(10) Pub. No.: US 2014/0170689 A1
(43) Pub. Date: Jun. 19, 2014

(54) Modified Diguanylate Cyclase-Phosphodiesterase and Method for Enzymatic Production of Cyclic-diGMP

(71) Applicants: Natasha M. NESBITT, Coram, NY (US); Roger A. JOHNSON, East Setauket, NY (US); Elizabeth M. BOON, Stony Brook, NY (US)

(72) Inventors: Natasha M. NESBITT, Coram, NY (US); Roger A. JOHNSON, East Setauket, NY (US); Elizabeth M. BOON, Stony Brook, NY (US)

(73) Assignee: The Research Foundation for The State University of New York, Albany, NY (US)

(21) Appl. No.: 14/104,488

(22) Filed: Dec. 12, 2013

Related U.S. Application Data
(60) Provisional application No. 61/736,329, filed on Dec. 12, 2012.

(51) Int. Cl. C12N 9/18; C12Q 1/44; C12P 19/36

(52) U.S. Cl. CPC C12N 9/18; C12P 19/36; C12Q 1/44
USPC 435/19; 435/188; 435/90

(57) ABSTRACT

The present invention provides a recombinant polypeptide comprising a first portion and a second portion, wherein the sequence of the first portion is fully identical to amino acids 1 to 248 of the sequence set forth as SEQ ID NO:1 and the sequence of the second portion is other than amino acids 249 to 511 of the sequence set forth as SEQ ID NO:1.

This application claims the benefit of U.S. Provisional Application No. 61/736,329, filed Dec. 12, 2012, the contents of which are hereby incorporated by reference in its entirety.

The invention was made with government support under Grant number N00014-10-1-0099 awarded by the Office of Naval Research. The government has certain rights in the invention.

This application incorporates-by-reference nucleotide and/or amino acid sequences which are present in the file named “131212_{—}2238_{—}84750_Sequence_Listing_LPT.txt,” which is 18 kilobytes in size, and which was created Dec. 11, 2013 in the IBM-PC machine format, having an operating system compatibility with MS-Windows, which is contained in the text file filed Dec. 12, 2013 as part of this application.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Cyclic bis(3′,5′)diguanylate monophosphate (cyclic-diGMP) is a secondary messenger that is involved in the regulation of various cellular processes. The nucleotide is ubiquitous in bacteria and regulates a range of functions including developmental transitions, aggregative behavior, adhesion, biofilm formation, and virulence factors (Ryan, R. P. et al. 2006). There is a growing interest in understanding the underlying mechanisms by which cyclic-diGMP regulates these processes. A better understanding of, for example, biofilm formation, bacterial resistance, and cell motility, would have implications in the medical and agricultural fields. However, the high cost of obtaining cyclic-diGMP ($260 for per μmol) has hampered research on the function of cyclic-diGMP.

Several groups have achieved chemical synthesis of cyclic-diGMP. However, these methods require multiple steps that involve protection and deprotection of various functional groups of GTP (Ross, P. et al (1990); Kawai, R. et al (2003); Hayakawa, Y. et al (2003); Zhang, Z. at al (2004). The enzymatic synthesis of cyclic-diGMP has also been reported by several groups using diguanylate cyclases. However, the enzymes used by these groups contain a product-inhibition site denoted by a conserved RxxD motif thereby limiting the amount of cyclic-diGMP that can be produced. One group reported generating a variant of a diguanylate cyclase from the thermophile Thermotoga maritime, encoded by the gene TM1788, in which they changed the RxxD motif to AxxD (Rao, F. et al. 2009). Despite making this variant they still observed product inhibition at high concentrations of GTP.

### SUMMARY OF THE INVENTION

The present invention provides a recombinant polypeptide comprising a first portion and a second portion, wherein the sequence of the first portion is fully identical to amino acids 1 to 248 of the sequence set forth as SEQ ID NO:1 and the sequence of the second portion is other than amino acids 249 to 511 of the sequence set forth as SEQ ID NO: 1.

The present invention provides a method of identifying a polypeptide which has diguanylate cyclase activity and which has no phosphodiesterase activity, the method comprising:
(i) mutating one or more amino acids at positions 249 to 511 of the polypeptide comprising amino acids whose sequence is set forth as SEQ ID NO:1 to obtain a mutant,
(ii) determining the diguanylate cyclase activity of the mutant, and
(iii) determining the phosphodiesterase activity of the mutant,
wherein the presence of diguanylate cyclase activity and a absence of phosphodiesterase activity identifies the polypeptide which has diguanylate cyclase activity and which has no phosphodiesterase activity.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Scheme of the reaction catalyzed by bi-functional diguanylate cyclase-phosphodiesterases containing the conserved GG(D/E)EF and ExL motifs.

FIG. 2. HPLC analysis of the reaction catalyzed by wild type Avi_{—}3097. The enzyme (1 μM) was incubated with 100 μm of GTP at 30° C. After 30 minutes of incubation the reaction was analyzed by HPLC using a reverse phase C18 column. NAD+ was used as an internal chromatography standard. The inset is the spectrum collected from MALDI analysis of the compound eluting at 16.4 min.

FIG. 3. Primary structure alignment of bi-functional diguanylate cyclase-phosphodiesterase enzymes. Conserved residues are highlighted by an asterisk in red. The PDE domain of Avi_{—}3097 is predicted to start at the arginine indicated by the arrow, which corresponds to residue 249 of the full-length protein, when its primary structure is analyzed with Prosite (http://prosite.expasy.org/).

FIG. 4. HPLC analysis of wild type and AVL reaction with GTP as substrate. Enzymes were incubated with 1 mM GTP overnight at room temperature. Samples are (from top to bottom), c-diGMP standard, pGpG standard, assay run with wild type enzyme and assay run with the AVL variant. Wild type enzyme was run as a control to show that the unaltered enzyme converts GTP to pGpG.

FIG. 5. Structural model of the putative PDE active site of Avi_{—}3097. The model was constructed using Modeller (http://salilab.org/modeller/) with the EAL domain of YkuI (pdb 2W27) serving as the template. The image was generated using VMD software (http://www.ks.uiuc.edu/Research/vmd/). Cyclic-diGMP is denoted in ball and stick form.

FIG. 6. Sequence listing of wild-type hi-functional DGC-PDE enzyme. Herein referred to as SEQ ID NO:1.

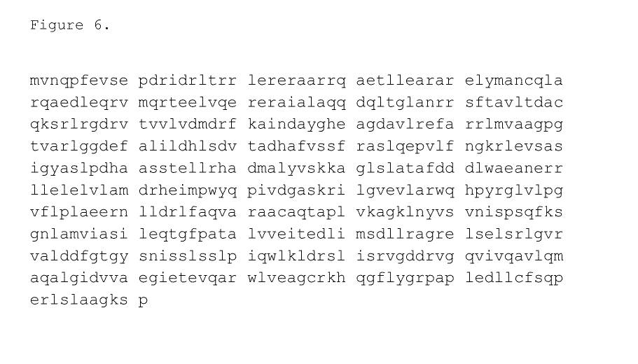

FIG. 7. Sequence listing of AVL variant. Herein referred to as SEQ ID NO:2.

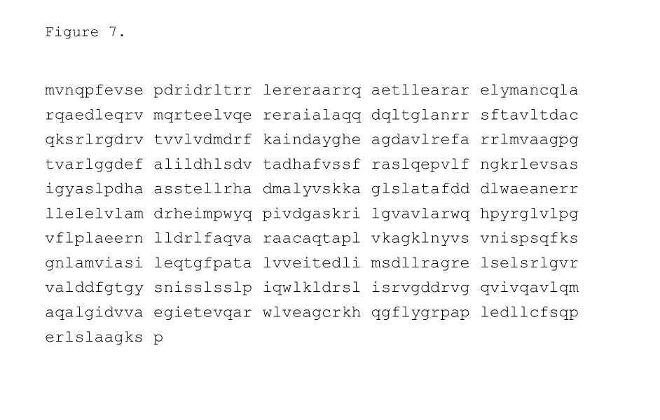

FIG. 8. Sequence listing of AAL variant. Herein referred to as SEQ ID NO:3.

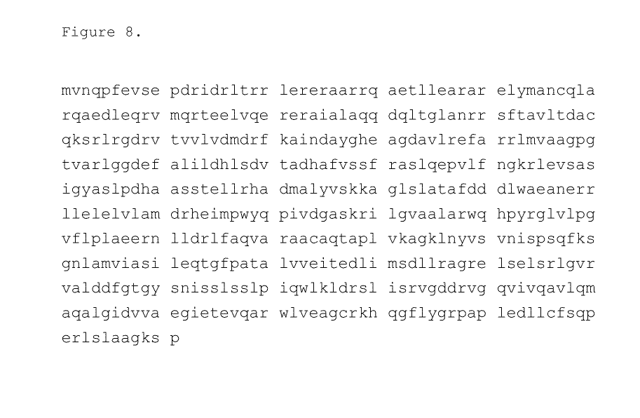

FIG. 9. Sequence listing of nucleotides that encodes the AVL variant. Herein referred to as SEQ ID NO:4.

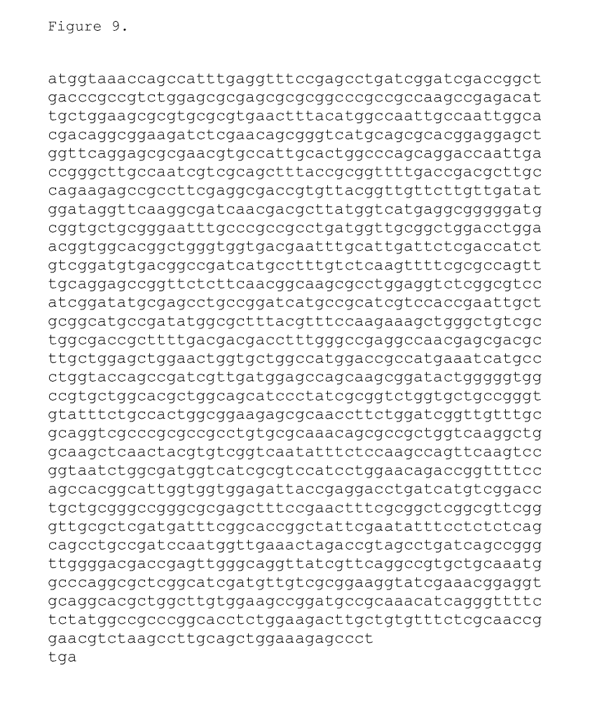

FIG. 10. Sequence listing of nucleotides that encodes the AAL variant. Herein referred to as SEQ ID NO:5.

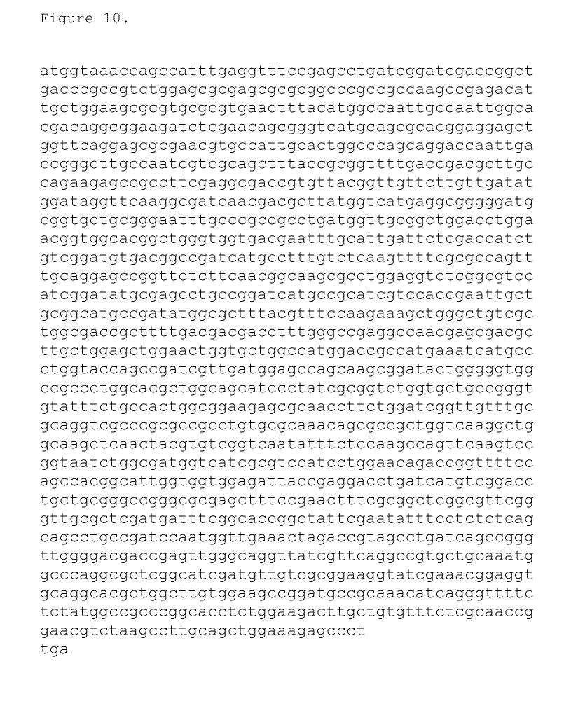

### DETAILED DESCRIPTION OF THE INVENTION

The present invention provides a recombinant polypeptide comprising a first portion and a second portion, wherein the sequence of the first portion is fully identical to amino acids 1 to 248 of the sequence set forth as SEQ ID NO:1 and the sequence of the second portion is other than amino acids 249 to 511 of the sequence set forth as SEQ ID NO:1.

In some embodiments, the polypeptide wherein the sequence of the second portion has at least 80% identity with amino acids 249 to 511 of the sequence set forth as SEQ ID NO:1.

In some embodiments, the polypeptide wherein the sequence of the second portion has at least 90% identity with amino acids 249 to 511 of the sequence set forth as SEQ ID NO:1.

In some embodiments, the polypeptide wherein the sequence of the second portion has at least 95% identity with amino acids 249 to 511 of the sequence set forth as SEQ ID NO:1.

In some embodiments, the polypeptide wherein the sequence of the second portion has at least 99% identity with amino acids 249 to 511 of the sequence set forth as SEQ ID NO:1.

In some embodiments, the polypeptide which is a substitution variant of the polypeptide whose sequence is set forth as SEQ ID NO:1.

In some embodiments, the polypeptide comprising an amino acid substitution at one or more, two or more positions, three or more positions, or four or more positions.

In some embodiments, the polypeptide wherein the variant contains no deletions.

In some embodiments, the polypeptide wherein the variant contains no insertions.

In some embodiments, the polypeptide wherein the variant contains a deletion or insertion.

In some embodiments, the polypeptide wherein the variant contains a deletion and insertion.

In some embodiments, the polypeptide wherein the amino acid substitution is a conservative substitution.

In some embodiments, the polypeptide comprising an amino acid substitution at one or more positions corresponding to positions E284, V285, F302, E307, N342, E374, E377, D404, D405, F406, S411, K425, E461, E464, or Q481.

In some embodiments, the polypeptide comprising an amino acid substitution at position E284.

In some embodiments, the polypeptide comprising an amino acid substitution at position E284 and V285.

In some embodiments, the polypeptide comprising an amino acid substitution at positions corresponding to positions E284, V285, F302, E307, N342, E374, E377, D404, D405, F406, S411, K425, E461, E464, or Q481.

In some embodiments, the polypeptide comprising a modification at amino acids 284, or 284 and 285.

In some embodiments, the polypeptide wherein the motif at amino acids 284-286 is xxL, wherein x represents any amino acid.

In some embodiments, the polypeptide wherein the modified EVL motif at amino acids 284-286 is xVL, wherein x represents any amino acid.

In some embodiments, the polypeptide wherein motif at amino acids 284-286 is AAL.

In some embodiments, the polypeptide wherein the motif at amino acids 284-286 is AVL.

In some embodiments, the polypeptide comprising the polypeptide of any one of claims 1-20, and one or more acceptable carriers.

In some embodiments, a composition for preparing a cyclic dinucleotide monophosphate comprising the polypeptide of any one of claims 1-20, and one or more acceptable carriers.

In some embodiments, a composition for preparing di-guanosine monophosphate (cyclic-diGMP) comprising the polypeptide of any one of claims 1-20, and one or more acceptable carriers.

In some embodiments, a cell comprising the polypeptide any one of claims 1-20.

In some embodiments, a process for the preparation of a cyclic dinucleotide monophosphate by enzymatic synthesis comprising:
contacting the polypeptide of the present invention with a purine nucleotide triphosphate and isolating the cyclic dinucleotide monophosphate.

In some embodiments, the process comprising the coupling of two purine nucleotide triphosphate molecules to form two molecules of pyrophosphate and the cyclic dinucleotide monophosphate.

In some embodiments, the process wherein the purine nucleotide triphosphate is guanosine triphosphate (GTP).

In some embodiments, a process for the preparation of a mixed cyclic dinucleotide monophosphate by enzymatic synthesis comprising:
contacting the polypeptide of the present invention with a purine nucleotide triphosphate in the presence of a second purine nucleotide triphosphate and isolating the mixed cyclic dinucleotide monophosphate.

In some embodiments, the process comprising the coupling of two different purine nucleotide triphosphate molecules to form two molecules of pyrophosphate and a mixed cyclic dinucleotide monophosphate.

A non-limiting example of a mixed cyclic dinucleotide monophosphate is

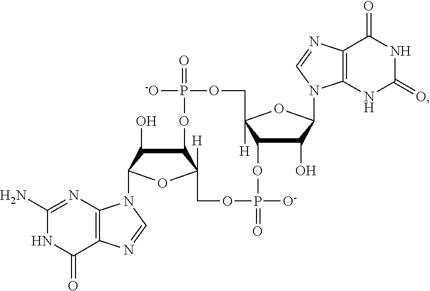

formed by the coupling of GTP and XTP.

In some embodiments, the purine nucleotide triphosphate or modified purine nucleotide triphosphate is selected from the group consisting of:
1-Thio-adenosine-5′-triphosphate (α-Thio-ATP),
1-Thio-guanosine-5′-triphosphate (α-Thio-GTP),
2′-Bromo-2′-deoxy-adenosine-5′-triphosphate (2′Br-2′dATP),
2′-Chloro-2′-deoxy-adenosine-5′-triphosphate (2′Cl-2′dATP),
2′-Deoxy-2,2′-difluoro-adenosine 5′-triphosphate (2,2′-diF-ATP),
2′-Fluoro-2′-deoxy-guanosine-5′-triphosphate (2′F-2′d-GTP),
2′-Fluoro-2′-deoxy-arabinosyl-guanosine-5′-triphosphate (2′F-2′d-Ara-GTP),
2′-Fluoro-2′-deoxy-xylosyl-guanosine-5′-triphosphate (2′F-2′d-Xylo-GTP)
2′-Iodo-2′-deoxy-adenosine-5′-triphosphate (2′I-2′dATP),
2′-Methylseleno-2′-deoxy-adenosine-5′-triphosphate (2′-MeSe-2′dATP),
2′-Amino-2′-deoxy-adenosine-5′-triphosphate (2′-Amino-2′dATP),
2-Amino-2′-deoxy-adenosine-5′-triphosphate (2-Amino-2′dATP),
2′-Amino-2′-deoxy-guanosine-5′-triphosphate (2′-Amino-2′dGTP),
2-Amino-6-Cl-purine-2′-deoxyriboside-triphosphate (2-Amino-6-Cl-purine-2′drTP),
2-Amino-6-Cl-purine-riboside-5′-triphosphate (2-Amino-6-Cl-purine-rTP),
2-Amino-adenosine-5′-triphosphate (2-Amino-ATP),
2-Amino-purine-2′-deoxyriboside-triphosphate (2-Aminopurine-2′drTP)
2-Amino-purine-riboside-5′-triphosphate (2-Aminopurine-rTP),
2′-Azido-2′-deoxy-adenosine-5′-triphosphate (2′-Azido-2′dATP),
2′-Azido-2′-deoxy-guanosine-5′-triphosphate (2′-Azido-2′dGTP),
2-Bromo-2′-deoxy-adenosine-5′-triphosphate (2Br-2′dATP),
2-Bromo-adenosine-5′-triphosphate (2Br-ATP),
2-Chloro-2′-deoxyadenosine-5′-triphosphate (2Cl-2′dATP)
2-Chloro-adenosine-5′-triphosphate (2Cl-ATP),
2′-Deoxy-adenosine-5′-O-(1-borano-triphosphate) (1-Borano-2′dATP),
2′-Deoxy-adenosine-5′-O-(1-thio-triphosphate) (α-Thio-2′dATP),
2′-Deoxy-adenosine-5′-triphosphate (2′dATP),
2′-Deoxy-guanosine-5′-O-(1-boranotriphosphate) (1-Borano-2′dGTP),
2′-Deoxy-guanosine-5′-O-(1-thiotriphosphate) (α-Thio-dGTP)
2′-Deoxy-guanosine-5′-triphosphate (2′dGTP),
2′-Deoxy-inosine-5′-triphosphate (2′dITP),
2′-Deoxy-L-adenosine-5′-triphosphate (2′d-L-ATP),
2′-Deoxy-L-guanosine-5′-triphosphate (2′d-L-GTP),
2′-Deoxy-L-inosine-5′-triphosphate (2′d-L-ITP),
2′-Deoxy-L-xanthosine-5′-triphosphate (2′d-L-XTP),
2′-Deoxy-P-nucleoside-5′-triphosphate (2′dPTP),
2′-Deoxy-xanthosine-5′-triphosphate (2′dXTP),
2′-Fluoro-2′-deoxy-adenosine-5′-triphosphate (2′F-2′dATP),
2′-Fluoro-2′-deoxyguanosine-5′-triphosphate (2′F-2′dGTP),
2-Fluoro-adenine-arabinosyl-5′-triphosphate (2F-Ara-ATP),
2-Fluoro-adenine-arabinosyl-5′-triphosphate (2F-Ara-ATP),
2-Fluoro-adenine-xylosyl-5′-triphosphate (2F-Xylo-ATP),
2-Fluoro-adenosine-5′-triphosphate (2F-ATP),
2-Hydroxy-adenosine-5′-triphosphate (20H-ATP),
2-Iodo-2′-deoxy-adenosine-5′-triphosphate (2I-2′dATP),
2-Iodo-adenosine-5′-triphosphate (2I-ATP),
2′-O-Methyl-2-aminoadenosine-5′-triphosphate (2′-O-Methyl-2-Amino-ATP),
2′-O-Methyl-adenosine-5′-triphosphate (2′-O-Methyl-ATP),
2′-O-Methyl-guanosine-5′-triphosphate (2′-O-Methyl-GTP),
2′-O-Methyl-inosine-5′-triphosphate (2′-O-Methyl-ITP),
5-Nitro-1-indolyl-2′-deoxyribose-5′-triphosphate (5-Nitro-1-Indolyl-2′d-rTP),
6-Benzylamino-purine-2′-deoxy-riboside-5′-triphosphate (6-benzyl-2′d-rTP)),
6-Chloro-guanosine-5′-triphosphate (6-Cl-GTP),
6-Chloro-purine-2′-deoxyriboside-5′-triphosphate (6-Cl-purine-2′d-rTP),
6-Chloro-purine-riboside-5′-triphosphate (6-Cl-purine-rTP),
6-Mercapto-purine-riboside-5′-triphosphate (6-SH-purine-rTP),
6-Methylthio-guanosine-5′-triphosphate (6-MeS-GTP),
6-Methylthio-inosine-5′-triphosphate (6-MeS-ITP),
6-Thio-2′-deoxyguanosine-5′-triphosphate (6-S-2′dGTP),
6-Thio-guanosine-5′-triphosphate (6-S-GTP),
7-Deaza-2′-deoxy-adenosine-5′-triphosphate (7-Deaza-2′dATP),
7-Deaza-2′-deoxy-guanosine-5′-triphosphate (7-Deaza-2′dGTP),
7-Deaza-7-bromo-2′-deoxy-adenosine-5′-triphosphate (7-Deaza-7-Br-2′dATP),
7-Deaza-7-Iodo-2′-deoxy-guanosine-5′-triphosphate (7-Deaza-7-I-2′dGTP),
7-Deaza-7-iodo-2′-deoxy-adenosine-5′-triphosphate (7-Deaza-7-I-2′dATP),
7-Deaza-7-propargylamino-2′-deoxy-adenosine-5′-triphosphate (7-Deaza-7-propargyl-2′dATP),
7-Deaza-7-propargylamino-2′-deoxy-guanosine-5′-triphosphate (7-Deaza-7-propargyl-2′dGTP),
7-Deaza-adenosine-5′-triphosphate (7-Deaza-ATP),
7-Deaza-guanosine-5′-triphosphate (7-Deaza-GTP),
7-methyl-guanosine-5′-triphosphate (7-Me-GTP),
8-[(4-Amino)butyl]-amino-adenosine-5′-triphosphate (8-4Aminobutyl-amino-ATP),
8-[(6-Amino)hexyl]-amino-adenosine-5′-triphosphate-biotin (8-4Aminobutyl-amino-ATP-biotin),
8-[(6-Amino)hexyl]-amino-guanosine-5′-triphosphate (8-6Aminohexyl-amino-GTP),
8-[(6-Amino)hexyl]-amino-guanosine-5′-triphosphate-biotin (8-6Aminohexyl-amino-GTP-biotin),
8-Aza-adenosine-5′-triphosphate (8-Aza-ATP),
8-Azido-2′-deoxy-adenosine-5′-triphosphate (8-Azido-2′dATP)
8-Azido-adenosine-5′-triphosphate (8-Azido-ATP),
8-Bromo-guanosine-5′-triphosphate (8-Br-GTP),
8-Bromo-2′-deoxy-adenosine-5′-triphosphate (8Br-2′dATP),
8-Bromo-2′-deoxy-adenosine-5′-triphosphate (8-Br-2′dATP),
8-Bromo-adenosine-5′-triphosphate (8-Br-ATP),
8-Chloro-2′-deoxy-adenosine-5′-triphosphate (8-Cl-2′dATP),
8-Chloro-adenosine-5′-triphosphate (8-Cl-ATP),
8-Iodo-guanosine-5′-triphosphate (8-I-GTP),
8-Oxo-2′-deoxy-adenosine-5′-triphosphate (8-Oxo-2′dATP),
8-Oxo-2′-deoxy-guanosine-5′-triphosphate (8-Oxo-2′dGTP),
8-Oxo-adenosine-5′-triphosphate (8-Oxo-ATP),
8-Oxo-guanosine-5′-triphosphate (8-Oxo-GTP),
Adenine-2′-deoxy-arabinosyl-5′-triphosphate (Ara-2′dATP),
Adenine-2′-deoxy-xylosyl-5′-triphosphate (Xylo-2′dATP),
Adenine-2′-fluoro-2′-deoxy-arabinosyl-5′-triphosphate (2′F-2′d-Ara-ATP),
Adenine-2′-fluoro-2′-deoxy-arabinosyl-5′-triphosphate (2′F-2′d-Ara-ATP),
Adenine-arabinosyl-5′-triphosphate (Ara-ATP),
Adenine-xylosyl-5′-triphosphate (Xylo-ATP),
Biotin-16-7-deaza-7-aminoallyl-2′-dGTP (Biotin-16-7-Deaza-AA-dGTP)
Ganciclovir triphosphate (9-(1,2-Dihydroxy-2-propoxymethyl)guanine-5′-Triphosphate, GCV-TP),
Guanine-2′-deoxy-xylosyl-5′-triphosphate (Xylo-2′dGTP),
Guanine-xylosyl-5′-triphosphate (Xylo-GTP),
Guanine-2′-deoxy-arabinosyl-5′-triphosphate (Ara-2′dGTP),
Guanine-2′-fluoro-2′-deoxy-arabinosyl-5′-triphosphate (2′F-2′d-Ara-GTP),
Guanine-arabinosyl-5′-triphosphate (Ara-GTP),
Hypoxanthine-2′-deoxy-arabinosyl-5′-triphosphate (Ara-2′dITP),
Hypoxanthine-2′-deoxy-xylosyl-5′-triphosphate (Xylo-2′dITP),
Hypoxanthine-arabinosyl-5′-triphosphate (Ara-ITP),
Hypoxanthine-xylosyl-5′-triphosphate (Xylo-ITP),
Inosine-5′-triphosphate (ITP)
N1-Methyl-adenosine-5′-triphosphate (N1-Methyl-ATP),
N1-Methyl-guanosine-5′-triphosphate (N1-Methyl-GTP),
N²-Methyl-2′-deoxy-guanosine-5′-triphosphate (N²-Methyl-dGTP),
N6-(4-Amino)butyl-2′-deoxy-adenosine-5′-triphosphate (N6-aminobutyl-2′dATP),
N6-(4-Amino)butyl-adenosine-5′-triphosphate (N6-aminobutyl-ATP),
N6-(6-Amino)hexyl-2′-deoxy-adenosine-5′-triphosphate (N6-aminohexyl-2′dATP),
N6-(6-Amino)hexyl-2′-deoxy-adenosine-5′-triphosphate-biotin (N6-aminohexyl-2′dATP-biotin),
N6-(6-Amino)hexyl-adenosine-5′-triphosphate (N6-aminohexyl-ATP),
N6-(6-Amino)hexyl-adenosine-5′-triphosphate-biotin (N6-aminohexyl-ATP-biotin),
N6-(6-Azido)hexyl-2′-deoxy-adenosine-5′-triphosphate (N6-azidohexyl-2′dATP),
N6-(6-Azido)hexyl-adenosine-5′-triphosphate (N6-azidohexyl-ATP),
N6-(6-Propargyl)-2′-deoxy-adenosine-5′-triphosphate (N6-propargyl-2′dATP),
N6-(6-Propargyl)-adenosine-5′-triphosphate (N6-propargyl-ATP),
N6-Benzyl-2′-deoxy-adenosine-5′-triphosphate (N6-benzyl-2′dATP),
N6-Benzyl-adenosine-5′-triphosphate (N6-benzyl-ATP),
N6-Methyl-2′-deoxy-adenosine-5′-triphosphate (N6-Methyl-2′dATP),
N6-Methyl-adenosine-5′-triphosphate (N6-Me-ATP),
O6-Methyl-2′-deoxy-guanosine-5′-triphosphate (O6-Methyl-dGTP),
O6-Methyl-guanosine-5′-triphosphate (O6-Methyl-GTP),
Xanthine-2′-deoxy-arabinosyl-5′-triphosphate (2′d-Ara-XTP),
Xanthine-2′-deoxy-xylosyl-5′-triphosphate (2′d-Xylo-XTP),
Xanthine-arabinosyl-5′-triphosphate (Ara-XTP),
Xanthine-xylosyl-5′-triphosphate (Xylo-XTP) and
Xanthosine-5′-triphosphate (XTP).

In some embodiments, a process for the preparation of cyclic di-guanosine monophosphate (cyclic-diGMP) by enzymatic synthesis comprising:
contacting the polypeptide of the present invention with guanosine triphosphate (GTP) and isolating the cyclic-diGMP.

In some embodiments, the process comprising the coupling of two guanosine triphosphate (GTP) molecules to form two molecules of pyrophosphate and the cyclic di-guanosine monophosphate (cyclic-diGMP).

In some embodiments, the process wherein the polypetide is immobilized.

In some embodiments, the process wherein the polypetide is immobilized to Ni-NTA resin.

In some embodiments, the process wherein the polypetide is immobilized to a support matrix.

In some embodiments, the process wherein the polypeptide is contacted with its substrate at a temperature in the range from 0 to 100° C.

In some embodiments, the process wherein the polypeptide is contacted with its substrate at a temperature in the range from 0 to 35° C.

In some embodiments, the process wherein the polypeptide is contacted with its substrate at a temperature of 30° C.

In some embodiments, the process wherein the polypeptide is contacted with its substrate at a pH range from 5 to 12.

In some embodiments, the process wherein the polypeptide is contacted with its substrate at a pH range from 7 to 8.

In some embodiments, the process wherein the polypeptide is contacted with its substrate at a pH of 7.5.

The present invention provides a method of identifying a polypeptide which has diguanylate cyclase activity and which has no phosphodiesterase activity, the method comprising:
(i) mutating one or more amino acids at positions 249 to 511 of the polypeptide comprising amino acids whose sequence is set forth as SEQ ID NO:1 to obtain a mutant,
(ii) determining the diguanylate cyclase activity of the mutant, and
(iii) determining the phosphodiesterase activity of the mutant,
wherein the presence of diguanylate cyclase activity and a absence of phosphodiesterase activity identifies the polypeptide which has diguanylate cyclase activity and which has no phosphodiesterase activity.

In some embodiments, the method identifying a polypeptide further comprising selecting the mutant with diguanylate cyclase activity and no phosphodiesterase activity, thereby producing the polypeptide which has diguanylate cyclase activity and which has no phosphodiesterase activity.

In some embodiments, a polypeptide produced by the method of the present invention.

In some embodiments, the polypeptide of the present invention has diguanylate cyclase activity.

In some embodiments, the polypeptide has of the present invention no phosphodiesterase activity.

In some embodiments, the polypeptide of the present invention has substantially similar diguanylate cyclase activity relative to the wild-type protein (SEQ ID NO:1) encoded by Avi_{—}3097 from Agrobacterium vitis strain S4. The term “Substantially similar diguanylate cyclase activity” as used above is intended to mean that similar amounts of the polypeptide of the present invention and wild-type protein would be needed to catalyze the conversion of 1 micro mole of substrate per minute.

In some embodiments, the polypeptide of the present invention has 100% diguanylate cyclase activity relative to the wild-type protein (SEQ ID NO:1) encoded by Avi_{—}3097 from Agrobacterium vitis strain 54. The term “100% diguanylate cyclase activity relative to the wild-type protein” as used above is intended to mean that the same amount of the polypeptide of the present invention relative to the wild-type protein would be needed to catalyze the conversion of 1 micro mole of substrate per minute.

In some embodiments, the polypeptide of the present invention has no substantial phosphodiesterase activity relative to the wild-type protein (SEQ ID NO:1) encoded by Avi_{—}3097 from Agrobacterium vitis strain S4. The term “No substantial phosphodiesterase activity” as used above is intended to mean that no amount of the polypeptide of the present invention relative to the wild-type protein would be capable of catalyzing the conversion of 1 micro mole of substrate per minute.

In some embodiments, the polypeptide of the present invention has 0-99% phosphodiesterase activity relative to the wild-type protein (SEQ ID NO:1) encoded by Avi_{—}3097 from Agrobacterium vitis strain S4.

In some embodiments, the polypeptide of the present invention has 50% phosphodiesterase activity relative to the wild-type protein (SEQ ID NO:1) encoded by Avi_{—}3097 from Agrobacterium vitis strain S4. The term “50% phosphodiesterase activity relative to the wild-type protein” as used above is intended to mean that twice the amount of the polypeptide of the present invention relative to the wild-type protein would be needed to catalyze the conversion of 1 micro mole of substrate per minute.

In some embodiments, the polypeptide of the present invention has 0% phosphodiesterase activity relative to the wild-type protein (SEQ ID NO:1) encoded by Avi_{—}3097 from Agrobacterium vitis strain S4. The term “0% phosphodiesterase activity relative to the wild-type protein” as used above is intended to mean that no amount of the polypeptide of the present invention relative to the wild-type protein would be capable of catalyzing the conversion of 1 micro mole of substrate per minute.

By any range disclosed herein, it is meant that all hundredth, tenth and integer unit amounts within the range are specifically disclosed as part of the invention. Thus, for example, 0-99% means that 0.02, 0.03 . . . 0.09%; 0.1, 0.2 . . . 0.9%; and 1, 2 . . . 99% are included as embodiments of this invention.

In some embodiments, the polypeptide of the present invention is a substantially purified polypeptide.

The present invention provides a polynucleotide that encodes the polypeptide of the present invention.

In some embodiments, the polynucleotide encodes variant AAL of the present invention.

In some embodiments, the polynucleotide encodes variant AVL of the present invention.

In some embodiments, an isolated and/or exogenous polynucleotide comprising a sequence of nucleotides encoding a polypeptide of the present invention.

In some embodiments, a polynucleotide of the present invention which is operably linked to a promoter capable of directing expression of the polynucleotide in a cell.

In some embodiments, a vector comprising a polynucleotide of the present invention.

In some embodiments, a host cell comprising a polynucleotide of the present invention.

In some embodiments, a host cell comprising a vector of the present invention.

In some embodiments, a host cell of the present invention which is a bacterial cell, yeast cell or a plant cell.

In some embodiments, an extract of the host cell and/or an organism, wherein the extract comprises a polypeptide of the present invention.

In some embodiments, A method of producing a polypeptide of the present invention, the method comprising cultivating a host cell or a vector under conditions which allow expression of the polynucleotide encoding the polypeptide, and recovering the expressed polypeptide.

In some embodiments, a composition comprising the polypeptide of the present invention, and one or more acceptable carriers.

In some embodiments, a composition for preparing a cyclic dinucleotide monophosphate comprising the polypeptide of the present invention, and one or more acceptable carriers.

In some embodiments, a composition for preparing di-guanosine monophosphate (cyclic-diGMP) comprising the isolated polypeptide of the present invention, and one or more acceptable carriers.

### General Techniques

The recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et at, Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), O. M. Glover and B. D. Hames (editors), DNA Cloning: A Practical Approach, Volumes 1-4, IRL Press (1995 and 1996), and F. M. Ausubel et al. (Editors), Current Protocols in Molecular Biology, Greene Pub. Associates and Wiley-Interscience (1988, including all updates until present).

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used herein, the phrase “at a position corresponding to amino acid number” refers to the relative position of the amino acid compared to surrounding amino acids. For example, in some embodiments a polypeptide of the invention may have additional N-terminal amino acids to assist with intracellular localization or extracellular secretion which alters the relative positioning of the amino acid when aligned against, for example, SEQ ID NO:1. In an embodiment, the polypeptide comprises the defined amino acid at the nominated residue number.

The term “biological material” is used herein in its broadest sense to include any product of biological origin. Such products include, but are not restricted to, food products for humans and animal feeds. The products include liquid media including water and liquid foodstuffs such as milk, as well as semi-solid foodstuffs such as yoghurt and the like. The present invention also extends to solid foodstuffs, particularly animal feeds. In an embodiment, it is preferred that the biological material is plant material such as, but not limited to, sugar cane, canola seeds, wheat seeds, barley seeds, sorghum seeds, rice, corn, pineapples, or cotton seeds.

As used herein, the term “extract” refers to any portion of a host cell or non-human transgenic organism of the invention. The portion may be a whole entity such as a seed of a plant, or obtained by at least partial homogenization and/or purification. This term includes portions secreted from the host cell, and hence encompasses culture supernatants.

### Amino Acids

“Amino acid,” “amino acid residue” and “residue” are used interchangeably herein to refer to an amino acid that is incorporated into a protein, polypeptide or peptide. The amino acid can be, for example, a naturally occurring amino acid or an analog of a natural amino acid that can function in a manner similar to that of the naturally occurring amino acid.

“C-terminal” and “N-terminal” amino acid, as used herein, refers to an amino acids at or in close proximity to the carboxy or amino terminal ends, respectively, of a given protein, protein domain or amino acid sequence motif such that no amino acid residue essential to the structure, function, or characterization of the protein, protein domain or amino acid sequence motif lie beyond said C-terminal amino acid or N-terminal amino acid.

### Polypeptides

By “substantially purified” or “purified” we mean a polypeptide that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated. However, at present there is no evidence that the polypeptides of the invention exist in nature.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered, preferably increased, amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, biologically active fragments, modifications, analogous and/or derivatives of the polypeptides described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the query sequence is at least 350 amino acids in length and the GAP analysis aligns the two sequences over a region of at least 350 amino acids. Even more preferably, the GAP analysis aligns the two sequences over their entire length.

As used herein a “biologically active fragment” is a portion of a polypeptide as described herein which maintains a defined activity of the full-length polypeptide. Biologically active fragments can be any size as long as they maintain the defined activity. Preferably, biologically active fragments are at least 300, more preferably at least 350, amino acids in length. Furthermore, biologically active means the ability to hydrolyse an organophoshate.

With regard to a defined polypeptide, it will be appreciated that % identity figures higher than those provided will encompass embodiments of the present invention. Thus, where applicable, in light of the minimum % identity figures, it is preferred that the polypeptide comprises an amino acid sequence which is at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of a polypeptide described herein can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide described herein can be subjected to in vitro mutagenesis. Such in vitro mutagenesis techniques may include sub-cloning the polynucleotide into a suitable vector, transforming the vector into a “mutator” strain such as the E. coli XL-1 red (Stratagene) and propagating the transformed bacteria for a suitable number of generations.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1.

In a preferred embodiment a mutant/variant polypeptide has one or two or three or four conservative amino acid changes when compared to a polypeptide specifically defined herein. Details of conservative amino acid changes are provided in Table 1. Preferably, if not specified otherwise, at a given amino acid position the polypeptide comprises an amino acid as found at the corresponding position of the polypeptide provided as SEQ ID NO:1.

If an amino acid at a nominated site is inconsistent with an amino acid substitution provided in Table 1, the nominated amino acid is preferred.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his; |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met, ala; phe |
| Lys (K) | arg |
| Met (M) | leu; phe; |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

Furthermore, if desired, unnatural amino acids or chemical amino acid analogues can be introduced as a substitution or addition into the polypeptide of the present invention. Such amino acids include. but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides of the present invention can be produced in a variety of ways, including production and recovery of natural proteins, production and recovery of recombinant proteins, and chemical synthesis of the proteins. In one embodiment, an isolated polypeptide of the present invention is produced by culturing a cell capable of expressing the polypeptide under conditions effective to produce the polypeptide, and recovering the polypeptide. Effective culture conditions include, but are not limited to, effective media, bioreactor, temperature, pH and oxygen conditions that permit protein production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins.

In an embodiment, a polypeptide of the invention comprises a signal sequence which is capable of directing secretion of the polypeptide from a cell. As the skilled person would appreciate, the signal sequence may or not be cleaved, or be partially cleaved, whilst being partially exported from the cell. However, when removing a signal sequence the cell may produce a heterogeneous population of polypeptides with slightly different, for example, N-terminal sequences. Thus, the term “consists of” encompasses such variants produced by the removal of signal sequences. A large number of such signal sequences have been isolated, which include N- and C-terminal signal sequences. Prokaryotic and eukaryotic N-terminal signal sequences are similar, and it has been shown that eukaryotic N-terminal signal sequences are capable of functioning as secretion sequences in bacteria. An example of such an N-terminal signal sequence is the bacterial β-lactamase signal sequence, which is a well-studied sequence, and has been widely used to facilitate the secretion of polypeptides into the external environment. An example of C-terminal signal sequences is the hemolysin A (hlyA) signal sequences of E. coli. Additional examples of signal sequences include, without limitation, aerolysin, alkaline phosphatase gene (phoA), chitinase, endochitinase, α-hemolysin, MIpB, pullulanase, Yops and a TAT signal peptide.

Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Preparation of Peptides and Polypeptides

Polypeptides may be produced via several methods, for example:

### 1) Synthetically:

Synthetic polypeptides can be made using a commercially available machine, using the known sequence of the desired protein or a portion thereof.

### 2) Recombinant Methods:

A preferred method of making the desired polypeptides of fragments thereof is to clone a polynucleotide comprising the cDNA of the desired gene or genomic DNA into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965; 23:1165-74 Appl Microbiol. 1967 July; 15(4):851-6; Can J Biochem. 1968 May; 46(5):441-4; Biochemistry. 1968 July; 7(7):2574-80; Arch Biochem Biophys. 1968 Sep. 10; 126(3):746-72; Biochem Biophys Res Commun. 1970 Feb. 20; 38(4):825-30).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986),

3) Purification from Natural Sources:

A desired polypeptide, or naturally occurring fragments thereof, can be purified from natural sources (such as tissues) using many methods known to one of ordinary skill in the art, such as for example: immuno-precipitation with an appropriate antibody, or matrix-bound affinity chromatography with any molecule known to bind the desired protein. Protein purification is practiced as is known in the art as described in, for example, Marshak et al., “Strategies for Protein Purification and Characterization. A laboratory course manual.” CSHL Press (1996).

“Expression vector” refers to a vector that has the ability to incorporate and express heterologous DNA fragments in a foreign cell. Many prokaryotic and eukaryotic expression vectors are known and/or commercially available. Selection of appropriate expression vectors is within the knowledge of those having skill in the art.

### Polynucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

The synthesis of any of the nucleic acids described herein is within the skills of the one of the art. Such synthesis is, among others, described in Beaucage S. L. and Iyer R. P., Tetrahedron 1992; 48: 2223-2311, Beaucage S. L. and Iyer R. P., Tetrahedron 1993; 49: 6123-6194 and Caruthers M. H. et. al., Methods Enzymol. 1987; 154: 287-313, the synthesis of thioates is, among others, described in Eckstein F., Annu. Rev. Biochem. 1985; 54: 367-402, the synthesis of RNA molecules is described in Sproat B., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

All analogues of, or modifications to, a polynucleotide may be employed with the present invention, provided that said analogue or modification does not substantially affect the function of the polynucleotide. The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, psuedo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogues of polynucleotides can be prepared wherein the structure of the nucleotide is fundamentally altered and that are better suited as therapeutic or experimental reagents. An example of a nucleotide analogue is a peptide nucleic acid (PNA) wherein the deoxyribose (or ribose) phosphate backbone in DNA (or RNA is replaced with a polyamide backbone which is similar to that found in peptides. PNA analogues have been shown to be resistant to degradation by enzymes and to have extended lives in vivo and in vitro. Further, PNAs have been shown to bind stronger to a complementary DNA sequence than a DNA molecule. This observation is attributed to the lack of charge repulsion between the PNA strand and the DNA strand. Other modifications that can be made to oligonucleotides include polymer backbones, cyclic backbones, or acyclic backbones.

### Polypeptide Modifications

The polypeptides employed in the present invention may also be modified, optionally chemically modified, in order to improve their enzymatic activity. “Chemically modified”—when referring to the polypeptides, means a polypeptide where at least one of its amino acid residues is modified either by natural processes, such as processing or other post-translational modifications, or by chemical modification techniques which are well known in the art. Among the numerous known modifications typical, but not exclusive examples include:

acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

Additional possible polypeptide modifications (such as those resulting from nucleic acid sequence alteration) include the following:

“Deletion”—is the change in either nucleotide or amino acid sequence in which one or more nucleotides or amino acid residues, respectively, are absent.

“Insertion” or “addition”—is the change in a nucleotide or amino acid sequence which has resulted in the addition of one or more nucleotides or amino acid residues, respectively, as compared to the naturally occurring sequence.

“Substitution”—is the replacement of one or more nucleotides or amino acids by different nucleotides or amino acids, respectively. As regards amino acid sequences the substitution may be, but are not limited to, conservative or non-conservative.

By “homolog/homology”, as utilized in the present invention, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci, USA 80:726); for instance, using a window size of 20 nucleotides, a word length of 4 nucleotides, and a gap penalty of 4, computer-assisted analysis and interpretation of the sequence data, including alignment, can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

“Having at least X % identity”—with respect to two amino acid or nucleotide sequences, refers to the percentage of residues that are identical in the two sequences when the sequences are optimally aligned. Thus, 90% amino acid sequence identity means that 90% of the amino acids in two or more optimally aligned polypeptide sequences are identical.

The terms “nucleic acid”, “polynucleotide” and “nucleic acid sequence” are used interchangeably herein, and each refers to a polymer of deoxyribonucleotides and/or ribonucleotides. The deoxyribonucleotides and ribonucleotides can be naturally occurring or synthetic analogues thereof. ‘Nucleic acid’ shall mean any nucleic acid, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA). Nucleic acids include, without limitation, anti-sense molecules and catalytic nucleic acid molecules such as ribozymes and DNAzymes. Nucleic acids also include nucleic acids coding for peptide analogs, fragments or derivatives which differ from the naturally-occurring forms in terms of the identity of one or more amino acid residues (deletion analogs containing less than all of the specified residues; substitution analogs wherein one or more residues are replaced by one or more residues; and addition analogs, wherein one or more resides are added to a terminal or medial portion of the peptide) which share some or all of the properties of the naturally-occurring forms.

Compositions of the present invention include excipients, also referred to herein as “acceptable carriers”. An excipient can be any material that the animal, plant, plant or animal material, or environment (including soil and water samples) to be treated can tolerate. Examples of such excipients include water, saline. Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal or o-cresol, formalin and benzyl alcohol. Excipients can also be used to increase the half-life of a composition, for example, but are not limited to, polymeric controlled release vehicles, biodegradable implants, liposomes, bacteria, viruses, other cells, oils, esters, and glycols.

### Recombinant Vectors

One embodiment of the present invention includes a recombinant vector, which comprises at least one isolated/exogenous polynucleotide of the invention inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises the polynucleotide(s) operably linked to an expression vector. The phrase operably linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors include any vectors that function (i.e., direct gene expression) in recombinant cells, including in bacterial, fungal, endoparasite, arthropod, animal, and plant cells. Vectors of the invention can also be used to produce the polypeptide in a cell-free expression system, such systems are well known in the art.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, nematode, plant or animal cells, such as, but not limited to, tac, lac, trp, trc, oxy-pro, omp/lpp, rrnB, bacteriophage lambda, bacteriophage T7. T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

### Host Cells

Another embodiment of the present invention includes a host cell transformed with one or more recombinant molecules described herein or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides described herein or can be capable of producing such polypeptides after being transformed with at least one polynucleotide molecule as described herein. Host cells of the present invention can be any cell capable of producing at least one protein defined herein, and include bacterial, fungal (including yeast), parasite, nematode, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Useful yeast cells include Pichia sp., Aspergillus sp. and Saccharomyces sp. Particularly preferred host cells are bacterial cells, yeast cells or plant cells.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

It will be noted that any notation of a nitrogen in structures throughout this application, when used without further notation, are intended to represent all isotopes of nitrogen, such as ¹⁴N or ¹⁵N. Furthermore, any compounds containing ¹⁴N or ¹⁵N may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

The compounds used in the method of the present invention may be prepared by techniques well know in organic synthesis and familiar to a practitioner ordinarily skilled in the art. However, these may not be the only means by which to synthesize or obtain the desired compounds.

Guanosine 5′-triphosphate tris salt is available as Catalog No. G9002 from Sigma-Aldrich (St. Loius, Mo., USA).

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5th Edition (2007), and references therein, which are incorporated by reference herein. However, these may not be the only means by which to synthesize or obtain the desired compounds.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 661-19).

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Bifunctional DGC-PDE Enzyme

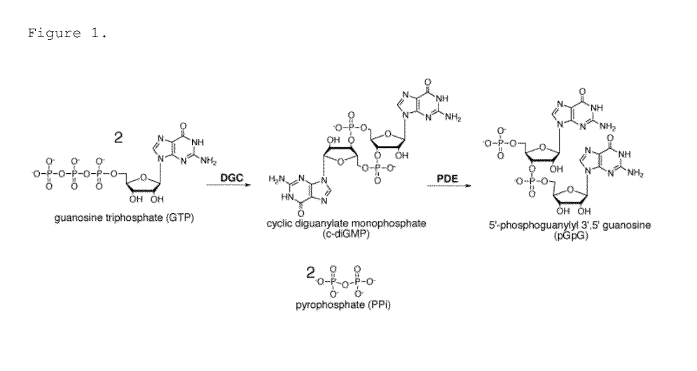

Based on its primary structure the protein encoded by the gene Avi_{—}3097 from Agrobacterium vitis strain S4 is predicted to be a bi-functional diguanylate cyclase-phosphodiesterase. Bi-functional diguanylate cyclase-phosphodiesterase enzymes are distinguished by their signature GG(D/E)EF and ExL conserved motifs, which catalyze diguanylate cyclase and phosphodiesterase activities, respectively (Tarutina, M. et al. 2006; Ferreira, R. B. et al. 2008; Kumar, M. et al. 2008; Gupta, K. et al. 2010). These hi-functional enzymes catalyze the formation of cyclic-diGMP and two molecules of pyrophosphate from two molecules of guanosine triphosphate (GTP) through their diguanylate cyclase (DGC) activity (Liu, N. et al. 2010; Levet-Paulo, M. et al. 2011). Cyclic-diGMP is hydrolyzed to 5′-phosphoguanylyl-(3′,5′)-guanosine (pGpG) through the enzyme's phosphodiesterase (PDE) activity (FIG. 1).

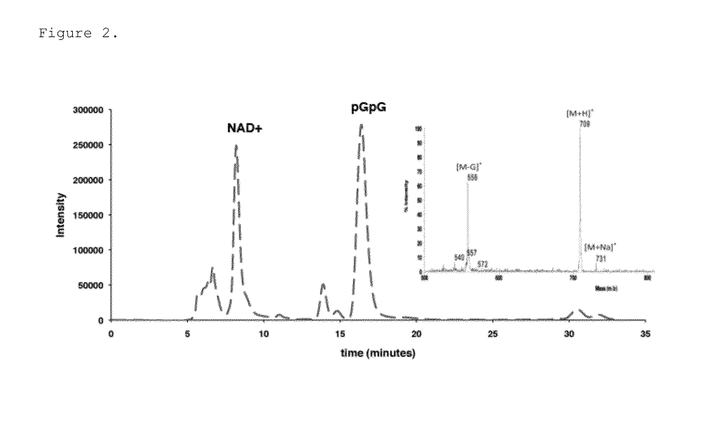

The gene Avi_{—}3097 from Agrobacterium vitis strain SA predicted to encode the wild type (naturally occurring) hi-functional DGC-PDE was cloned into the vector pET20b between the Nde1 and Xho1 restriction sites. Cloning between these two sites allowed for the production of protein containing a hexa-histidine appendage at its C-terminus. Clones of the gene containing either a hexa-histidine appendage or a glutathione S-transferase tag at the N-terminus are also available. The encoded protein was over-expressed in 2XYT medium by the addition of 10 μM isopropyl β-D-1-thiogalactopyranoside (IPTG) for 16 hours at 18° C. Escherichia coli BL21DE3pLysS competent cells served as host for protein over-expression. The over-expressed protein was purified by use of immobilized affinity chromatography (IMAC) with Ni-nitrilotriacetic acid (Ni-NTA) agarose (Qiagen) as the matrix. The purified protein was assayed using GTP as the substrate and was shown to produce pGpG when analyzed by high performance liquid chromatography (HPLC) and matrix-assisted laser. desorption ionization (MALDI) mass spectrometry (FIG. 2). After a 30-minute incubation at 30° C. we did not observe GTP present in our reaction mixture when it was analyzed by HPLC, indicating that all of the GTP had been consumed. The compound eluting with a retention time of 16.4, consistent with that of authentic pGpG, was collected and analyzed by MALDI with alpha-cyano-4-hydroxycinnamic acid as the matrix. The predominant species has a mass of 709 Daltons corresponding to the mass of pGpG plus a proton. These experiments confirm that the wild type enzyme encodes both diguanylate cyclase and phosphodiesterase activities.

### Example 2

### Variants Lacking PDE Activity

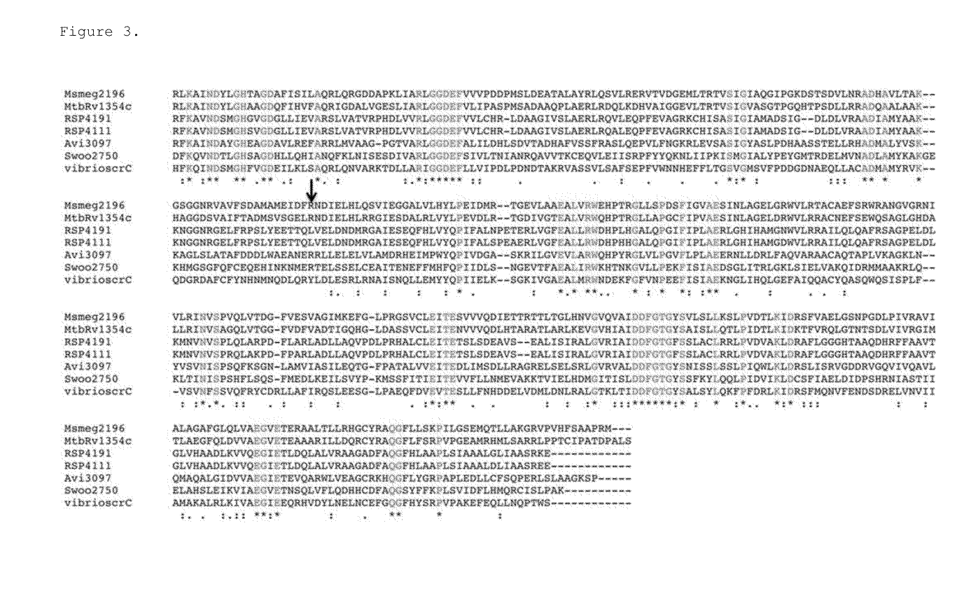

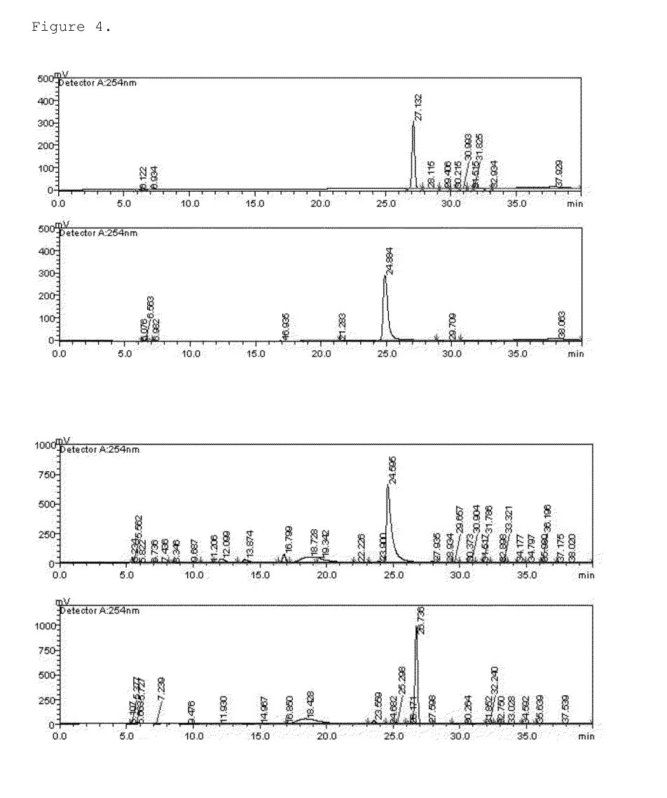

A primary structure alignment of bi-functional DGC-PDE enzymes reveals that there are a number of conserved amino acids that could be important for PDE activity via acid-base catalysis and/or substrate binding (FIG. 3). Using protein engineering in a manner similar to that described by the QuikChange™ Site-Directed Mutagenesis kit by Stratagene (now Agilent), several variants of the enzyme that lacked phosphodiesterase activity were generated. Starting with the signature PDE ExL motif, EVL for the protein encoded by Avi_{—}3097, known to be essential for phosphodiesterase activity, we generated AAL and AVL variants. These variants were expressed and purified by the same procedure as was the wild type enzyme. The overnight incubation of the full-length variant proteins with GTP resulted in the accumulation of cyclic-diGMP; no pGpG was formed (FIG. 4). This experiment showed that as predicted these variants lack phosphodiesterase activity.

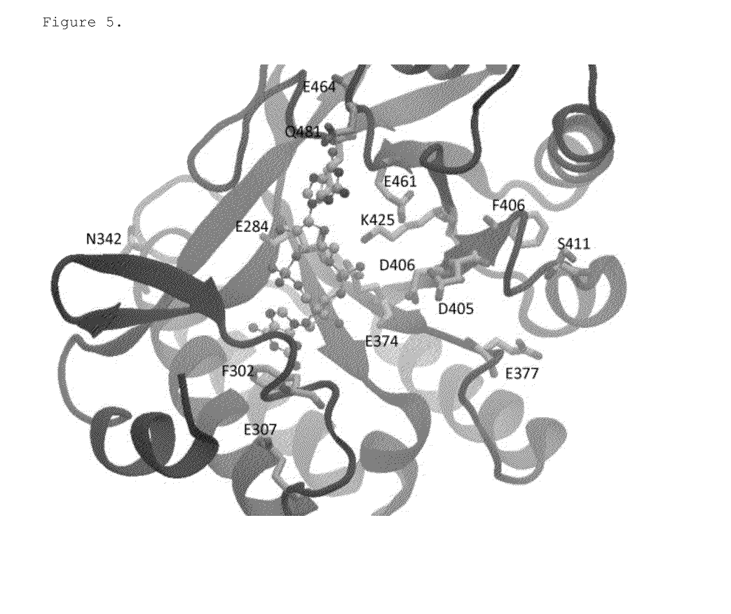

A structural model of the ExL domain of Avi_{—}3097 was generated using the ExL domain of YkuI (pdb 2W27) as the template and observed that a number of the conserved residues are located within close proximity to the predicted binding site of cyclic-diGMP (FIG. 5). Based on this model and studies conducted on previously characterized phosphodiesterases (Rao, F. et al. 2008; MInasov, G. et al. 2009; Rao, F. et al. 2009) it was hypothesized that these conserved residues are important for phosphodiesterase activity. To test this, several variants of the protein were generated changing conserved residues to alanine to remove the functional group. Again these variants were expressed and purified with the same procedure as was the wild type enzyme. These variants were assayed for phosphodiesterase activity using GTP as the substrate and observed an accumulation of cyclic-diGMP showing that several of the conserved residues are essential for phosphodiesterase activity.

The generation of the following variants of SEQ ID NO:1 resulted in ablation of phosphodiesterase activity:

PDE Ext. Motif

AVL: E284x (where “x” is alanine)

### PDE ExL Motif

AAL: E284x and E285x (where “x” is alanine).

### Example 3

### Additional Variants Lacking PDE Activity

The generation of the following variants results in ablation of phosphodiesterase activity (where “x” is any amino acid): F302x, E307x, N342x, E374x, E377x, D404x, D405x, F406x, S411x, K425x, E461x, E464x, Q481x and a truncated variant containing residues 1-248 (encoding the DGC domain of the bi-functional enzyme).

### Example 4

### Enzymatic Synthesis of Cyclic-diGMP

The AVL variant was used to run a large-scale synthesis of cyclic-diGMP from GTP. The reactions contained 9.9 nmoles of pure AVL, 5 μmoles of GTP, 50 mM of Tris-HCl, pH 7.5, 5 mM MgCl₂ in a final reaction volume of 2 mL at 30° C. At various time points 100 μL aliquots of the reaction were removed and boiled to denature the enzyme. The samples were centrifuged to remove precipitated enzyme and the resulting supernatant fractions were filtered and analyzed by HPLC on a C18 column. After 4 hours, the production of 1.9 μmoles cyclic-diGMP corresponding to a 76% conversion of GTP to cyclic-diGMP was observed. After 24 hours of incubation, 2.4 μmoles of cyclic-diGMP was produced corresponding to 96% conversion.

### Example 5

### Enzymatic Synthesis of Other Cyclic Dinucleotide Monophosphates

The isolated polypeptide variants described herein are used to synthesize various cyclic dinucleotide monophosphates from purine nucleotide triphosphates or modified purine nucleotide triphosphates. The enzymatic synthesis described herein is used to synthesize various cyclic dinucleotide monophosphates from purine nucleotide triphosphates or modified purine nucleotide triphosphates.

### Example 6

### Immobilized Polypeptide

In addition to the large scale in solution synthesis of cyclic-diGMP cyclic-diGMP has been synthesized using the AAL variant immobilized to Ni-NTA. The resin was saturated with protein and GTP, MgCl2 and Tris-HCl, pH 7.5, were added to the resin. The resin was slightly agitated to ensure proper mixing of substrate with enzyme. After a 30 min incubation at room temperature the column was washed with Tris-HCl, pH 7.5 to remove GTP and/or cyclic-diGMP. Conversion of GTP to cyclic-diGMP was observed. The polypeptide variants AAL and AVL are immobilized to any one of various known resins and the resins are used in the enzymatic synthesize of cyclic-diGMP from GTP. Any of the polypeptide variants described herein, immobilized to Ni-NTA or any other appropriate matrix, are used to synthesize cyclic-diGMP from GTP. Any of the polypeptide variants described herein, immobilized to Ni-NTA or any other appropriate matrix, are used to synthesize various cyclic dinucleotide monophosphates from purine nucleotide triphosphates, modified purine nucleotide triphosphates, or mixtures thereof.

### Example 7

### Radio-labeled Cyclic-diGMP

The AAL variant was used to generate radio-labeled cyclic-diGMP from α-³²P-GTP. The variant AAL and AVL are to generate labeled cyclic-diGMP from labeled GTP including the synthesis of: ³H-cyclic-diGMP, deuterated cyclic-diGMP, ¹⁴C-cyclic-diGMP, ¹³C-cyclic-diGMP, ¹³C, ¹⁵N-cyclic-diGMP, ¹⁵N-cyclic-diGMP, ³²P-cyclic-diGMP and ³³P-cyclic-diGMP or a mixture (i.e. compounds containing more than one label) of these cyclic-di-GMP molecules. The isolated polypeptide variants described herein are used to synthesize various radio-labeled cyclic dinucleotide monophosphates from radio-labeled purine nucleotide triphosphates or radio-labeled modified purine nucleotide triphosphates. The enzymatic synthesis described herein is used to synthesize various radio-labeled cyclic dinucleotide monophosphates from radio-labeled purine nucleotide triphosphates or radio-labeled modified purine nucleotide triphosphates.

### DISCUSSION

Cyclic bis(3′,5′)diguanylate monophosphate (cyclic-diGMP) is a secondary messenger involved in the regulation of various cellular processes that have medical and agricultural implications including biofilm formation, regulation of virulence factors, and cell mobility. There is a growing interest in the underlying mechanisms by which cyclic-diGMP regulates these processes. However, these experiments have been hampered mainly due to the high financial burden for obtaining large amounts of c-diGMP ($260 for per μmole from Biolog, Germany, Cat. No. C057). This hurdle was overcome by enzymatically synthesizing large quantities of cyclic-diGMP using variants of a bi-functional diguanylate cyclase-phosphodiesterase enzyme. Radioactive isotopes from the guanosine triphosphate (GTP) were also used to synthesize radio-labeled cyclic-diGMP using the variants of the bi-functional diguanylate cyclase-phosphodiesterase enzyme. The enzymatic synthesis allowed for the production of ˜$630 worth of cyclic-diGMP from only $18.30 worth of GTP.

The enzymatic synthesis of cyclic-diGMP has been reported by several groups using diguanylate cyclases. However, the enzymes used by these groups contain a product-inhibition site denoted by a conserved RxxD motif thereby limiting the amount of cyclic-diGMP produced. One group reported generating a variant of a diguanylate cyclase from the thermophile Thermotoga maritime, encoded by the gene TM1788, in which they changed the RxxD motif to AxxD. Despite making this variant they still observed product inhibition at high concentrations of GTP. Wild type Avi_{—}3097 and phosphodiesterase variants disclosed herein lack the conserved RxxD motif and do not exhibit product inhibition. Large amounts of cyclic-diGMP from GTP are enzymatically synthesized.

Under the experimental conditions disclosed herein, 2.4 μmole of cyclic-diGMP was produced from 5 μmole GTP, corresponding to a 96% conversion using 9.9 nmole of enzyme. Researchers reporting the improved enzymatic synthesis of cyclic-diGMP failed to calculate their conversions (Rao, F. and Pasunooti, S. et al. 2009; Zahringer, F. et al. 2010). However, the enzymes used resulted in product inhibition by cyclic-diGMP, which is not observed using the protein variants disclosed herein. Additionally, the variants retain enzymatic activity for several months making them ideal for long term storage and repeated cyclic-diGMP syntheses.

### REFERENCES

Ferreira, R. B., Antunes, L. C. Greenberg, E. P., and McCarter, L. L. (2008) Vibrio parahaemolyticus ScrC modulates cyclic dimeric GMP regulation of gene expression relevant to growth on surfaces, J Bacteriol 190, 851-860.
Gupta, K., Kumar, P., and Chatterji, D. (2010) Identification, activity and disulfide connectivity of C-di-GMP regulating proteins in Mycobacterium tuberculosis, PLoS One 5, e15072.
Hayakawa, Y., Nagata, R., Hirata, A. Hyodo, M., and Kawai, R. (2003) A facile synthesis of cyclic bis(3′-->5′)diguanylic acid.
Kawai, R., Nagata, R., Hirata, A., and Hayakawa, Y. (2003) A new synthetic approach to cyclic bis(3′-->5′)diguanylic acid, Nucleic Acids Res Suppl, 103-104.
Kumar, M., and Chatterji, D. (2008) Cyclic di-GMP: a second messenger required for long-term survival, but not for biofilm formation, in Mycobacterium smegmatis, Microbiology 154, 2942-2955.
Levet-Paulo, M., Lazzaroni, J. C., Gilbert, C., Atlan, D. Doublet, P., and Vianney, A. (2011) The atypical two-component sensor kinase Lp10330 from Legionella pneumophila controls the bifunctional diguanylate cyclase-phosphodiesterase Lp10329 to modulate bis-(3′-5)-cyclic dimeric GMP synthesis, J Biol Chem 286, 31136-31144.
Liu, N., Pak, T., and Boon, E. M. (2010) Characterization of a diguanylate cyclase from Shewanella woodyi with cyclase and phosphodiesterase activities, Mol Biosyst 6, 1561-1564.
Minasov, G., Padavattan, S., Shuvalova, L., Brunzelle, J. S., Miller, D. J., Basle, A., Massa, C., Collart, F. R., Schirmer, T., and Anderson, W. F. (2009) Crystal structures of YkuI and its complex with second messenger cyclic Di-GMP suggest catalytic mechanism of phosphodiester bond cleavage by EAL domains, J Biol Chem 284, 13174-13184
Rao, F., Yang, Y., Qi, Y., and Liang, Z. X. (2008) Catalytic mechanism of cyclic di-GMP-specific phosphodiesterase: a study of the EAL domain-containing RocR from Pseudomonas aeruginosa, J Bacteriol 190, 3622-3631.
Rao, F. et al. (2009) The functional role of a conserved loop in EAL domain-based cyclic di-GMP-specific phosphodiesterase, J Bacteriol 191, 4722-4731.
Rao, F. et al. (2009) Enzymatic synthesis of c-di-GMP using a thermophilic diguanylate cyclase, Anal Biochem 389, 138-142.
Ross, P. et al. (1990) The cyclic diguanylic acid regulatory system of cellulose synthesis in Acetobacter xylinum. Chemical synthesis and biological activity of cyclic nucleotide dimer, trimer, and phosphothioate derivatives, J Biol Chem 265, 18933-18943.
Ryan, R. P. et al. (2006) Cyclic Di-GMP Signaling in Bacteria: Recent Advances and New Puzzles, Journal of Bacteriology, 199, 8327-8334.
Tarutina, M., Ryjenkov, D. A., and Gomelsky, M. (2006) An unorthodox bacteriophytochrome from Rhodobacter sphaeroides involved in turnover of the second messenger c-di-GMP, J Biol Chem 281, 34751-34758.
Zahringer, F., Massa, C., and Schirmer, T. (2010) Efficient enzymatic production of the bacterial second messenger c-di-GMP by the diguanylate cyclase YdeH from E. coli, Appl Biochem Biotechnol 163, 71-79.
Zhang, Z., Gaffney, B. L., and Jones, R. A. (2004) c-di-GMP displays a monovalent metal ion-dependent polymorphism, J Am Chem Soc 126, 16700-16701.

1. A recombinant polypeptide comprising a first portion and a second portion,
wherein the sequence of the first portion is fully identical to amino acids 1 to 248 of the sequence set forth as SEQ ID NO:1 and the sequence of the second portion is other than amino acids 249 to 511 of the sequence set forth as SEQ ID NO:1.

2. The polypeptide of claim 1, wherein the sequence of the second portion has at least 80% identity with amino acids 249 to 511 of the sequence set forth as SEQ ID NO:1, or the sequence of the second portion has at least 90% identity with amino acids 249 to 511 of the sequence set forth as SEQ ID NO:1, or the sequence of the second portion has at least 95% identity with amino acids 249 to 511 of the sequence set forth as SEQ ID NO:1, or the sequence of the second portion has at least 99% identity with amino acids 249 to 511 of the sequence set forth as SEQ ID NO:1.

3-5. (canceled)

6. The polypeptide of claim 2, which is a substitution variant of the polypeptide whose sequence is set forth as SEQ ID NO:1.

7. The polypeptide of claim 6, comprising an amino acid substitution at one or more, two or more positions, three or more positions, or four or more positions.

8. The polypeptide of claim 6, wherein the variant contains no deletions or insertions.

9-10. (canceled)

11. The polypeptide of claim 6, wherein the amino acid substitution is a conservative substitution.

12. The isolated polypeptide of claim 6, comprising an amino acid substitution at one or more positions corresponding to positions E284, V285, F302, E307, N342, E374, E377, D404, D405, F406, S411, K425, E461, E464, or Q481.

13. The polypeptide of claim 12, comprising an amino acid substitution at position E284, or an amino acid substitution at position E284 and V285.

14. (canceled)

15. The polypeptide of claim 12, comprising an amino acid substitution at positions corresponding to positions E284, V285, F302, E307, N342, E374, E377, D404, D405, F406, S411, E425, E461, E464, or Q481.

16. The polypeptide of claim 12, comprising a modification at amino acids 284, or 284 and 285.

17. The polypeptide of claim 16, wherein the motif at amino acids 284-286 is xxL,
wherein x represents any amino acid.

18. (canceled)

19. The polypeptide of claim 17, wherein the motif at amino acids 284-286 is AAL or AVL.

20. (canceled)

21. A composition comprising the polypeptide of claim 1, and one or more acceptable carriers.

22. A composition for preparing a cyclic dinucleotide monophosphate or for preparing di-guanosine monophosphate (cyclic-diGMP) comprising the polypeptide of claim 1, and one or more acceptable carriers.

23-24. (canceled)

25. A process for the preparation of a cyclic dinucleotide monophosphate by enzymatic synthesis comprising:
contacting the polypeptide of claim 1 with a purine nucleotide triphosphate and isolating the cyclic dinucleotide monophosphate.

26. (canceled)

27. The process of claim 25, wherein the purine nucleotide triphosphate is guanosine triphosphate (GTP).

28. The process of claim 25, wherein the purine nucleotide triphosphate is selected from the group consisting of:
1-Thio-adenosine-5′-triphosphate (α-Thio-ATP),
1-Thio-guanosine-5′-triphosphate (α-Thio-GTP),
2′-Bromo-2′-deoxy-adenosine-5′-triphosphate (2′Br-2′dATP),
2′-Chloro-2′-deoxy-adenosine-5′-triphosphate (2′Cl-2′dATP),
2′-Deoxy-2,2′-difluoro-adenosine 5′-triphosphate (2,2′-diF-ATP),
2′-Fluoro-2′-deoxy-guanosine-5′-triphosphate (2′F-2′d-GTP),
2′-Fluoro-2′-deoxy-arabinosyl-guanosine-5′-triphosphate (2′F-2′d-Ara-GTP),
2′-Fluoro-2′-deoxy-xylosyl-guanosine-5′-triphosphate (2′F-2′d-Xylo-GTP)
2′-Iodo-2′-deoxy-adenosine-5′-triphosphate (2′I-2′dATP),
2′-Methylseleno-2′-deoxy-adenosine-5′-triphosphate (2′-MeSe-2′dATP),
2′-Amino-2′-deoxy-adenosine-5′-triphosphate (2′-Amino-2′dATP),
2-Amino-2′-deoxy-adenosine-5′-triphosphate (2-Amino-2′dATP),
2′-Amino-2′-deoxy-guanosine-5′-triphosphate (2′-Amino-2′dGTP),
2-Amino-6-Cl-purine-2′-deoxyriboside-triphosphate (2-Amino-6-Cl-purine-2′drTP),
2-Amino-6-Cl-purine-riboside-5′-triphosphate (2-Amino-6-Cl-purine-rTP),
2-Amino-adenosine-5′-triphosphate (2-Amino-ATP),
2-Amino-purine-2′-deoxyriboside-triphosphate (2-Aminopurine-2′drTP)
2-Amino-purine-riboside-5′-triphosphate (2-Aminopurine-rTP),
2′-Azido-2′-deoxy-adenosine-5′-triphosphate (2′-Azido-2′dATP),
2′-Azido-2′-deoxy-guanosine-5′-triphosphate (2′-Azido-2′dGTP),
2-Bromo-2′-deoxy-adenosine-5′-triphosphate (2Br-2′dATP),
2-Bromo-adenosine-5′-triphosphate (2Br-ATP),
2-Chloro-2′-deoxyadenosine-5′-triphosphate (2Cl-2′dATP)
2-Chloro-adenosine-5′-triphosphate (2Cl-ATP),
2′-Deoxy-adenosine-5′-O-(1-borano-triphosphate) (1-Borano-2′dATP),
2′-Deoxy-adenosine-5′-O-(1-thio-triphosphate) (α-Thio-2′dATP),
2′-Deoxy-adenosine-5′-triphosphate (2′dATP),
2′-Deoxy-guanosine-5′-O-(1-boranotriphosphate) (1-Borano-2′dGTP),
2′-Deoxy-guanosine-5′-O-(1-thiotriphosphate) (α-Thio-dGTP)
2′-Deoxy-guanosine-5′-triphosphate (2′dGTP),
2′-Deoxy-inosine-5′-triphosphate (2′dITP),
2′-Deoxy-L-adenosine-5′-triphosphate (2′d-L-ATP),
2′-Deoxy-L-guanosine-5′-triphosphate (2′d-L-GTP),
2′-Deoxy-L-inosine-5′-triphosphate (2′d-L-ITP),
2′-Deoxy-L-xanthosine-5′-triphosphate (2′d-L-XTP),
2′-Deoxy-P-nucleoside-5′-triphosphate (2′dPTP),
2′-Deoxy-xanthosine-5′-triphosphate (2′dXTP),
2′-Fluoro-2′-deoxy-adenosine-5′-triphosphate (2′F-2′dATP),
2′-Fluoro-2′-deoxyguanosine-5′-triphosphate (2′F-2′dGTP),
2-Fluoro-adenine-arabinosyl-5′-triphosphate (2F-Ara-ATP),
2-Fluoro-adenine-arabinosyl-5′-triphosphate (2F-Ara-ATP),
2-Fluoro-adenine-xylosyl-5′-triphosphate (2F-Xylo-ATP),
2-Fluoro-adenosine-5′-triphosphate (2F-ATP),
2-Hydroxy-adenosine-5′-triphosphate (20H-ATP),
2-Iodo-2′-deoxy-adenosine-5′-triphosphate (2I-2′dATP),
2-Iodo-adenosine-5′-triphosphate (2I-ATP),
2′-O-Methyl-2-aminoadenosine-5′-triphosphate (2-O-Methyl-2-Amino-ATP),
2′-O-Methyl-adenosine-5′-triphosphate (2′-O-Methyl-ATP),
2′-O-Methyl-guanosine-5′-triphosphate (2′-O-Methyl-GTP),
2′-O-Methyl-inosine-5′-triphosphate (2′-O-Methyl-ITP),
5-Nitro-1-indolyl-2′-deoxyribose-5′-triphosphate (5-Nitro-1-Indolyl-2′d-rTP),
6-Benzylamino-purine-2′-deoxy-riboside-5′-triphosphate (6-benzyl-2′d-rTP)),
6-Chloro-guanosine-5′-triphosphate (6-Cl-GTP),
6-Chloro-purine-2′-deoxyriboside-5′-triphosphate (6-Cl-purine-2′d-rTP),
6-Chloro-purine-riboside-5′-triphosphate (6-Cl-purine-rTP),
6-Mercapto-purine-riboside-5′-triphosphate (6-SH-purine-rTP),
6-Methylthio-guanosine-5′-triphosphate (6-MeS-GTP),
6-Methylthio-inosine-5′-triphosphate (6-MeS-ITP),
6-Thio-2′-deoxyguanosine-5′-triphosphate (6-S-2′dGTP),
6-Thio-guanosine-5′-triphosphate (6-S-GTP),
7-Deaza-2′-deoxy-adenosine-5′-triphosphate (7-Deaza-2′dATP),
7-Deaza-2′-deoxy-guanosine-5′-triphosphate (7-Deaza-2′dGTP),
7-Deaza-7-bromo-2′-deoxy-adenosine-5′-triphosphate (7-Deaza-7-Br-2′dATP),
7-Deaza-7-Iodo-2′-deoxy-guanosine-5′-triphosphate (7-Deaza-7-I-2′dGTP),
7-Deaza-7-iodo-2′-deoxy-adenosine-5′-triphosphate (7-Deaza-7-I-2′dATP),
7-Deaza-7-propargylamino-2′-deoxy-adenosine-5′-triphosphate (7-Deaza-7-propargyl-2′dATP),
7-Deaza-7-propargylamino-2′-deoxy-guanosine-5′-triphosphate (7-Deaza-7-propargyl-2′dGTP),
7-Deaza-adenosine-5′-triphosphate (7-Deaza-ATP),
7-Deaza-guanosine-5′-triphosphate (7-Deaza-GTP),
7-methyl-guanosine-5′-triphosphate (7-Me-GTP),
8-[(4-Amino)butyl]-amino-adenosine-5′-triphosphate (8-4Aminobutyl-amino-ATP),
8-[(6-Amino)hexyl]-amino-adenosine-5′-triphosphate-biotin (8-4Aminobutyl-amino-ATP-biotin),
8-[(6-Amino)hexyl]-amino-guanosine-5′-triphosphate (8-6Aminohexyl-amino-GTP),
8-[(6-Amino)hexyl]-amino-guanosine-5′-triphosphate-biotin (8-6Aminohexyl-amino-GTP-biotin),
8-Aza-adenosine-5′-triphosphate (8-Aza-ATP),
8-Azido-2′-deoxy-adenosine-5′-triphosphate (8-Azido-2′dATP)
8-Azido-adenosine-5′-triphosphate (8-Azido-ATP),
8-Bromo-guanosine-5′-triphosphate (8-Br-GTP),
8-Bromo-2′-deoxy-adenosine-5′-triphosphate (8Br-2′dATP),
8-Bromo-2′-deoxy-adenosine-5′-triphosphate (8-Br-2′dATP),
8-Bromo-adenosine-5′-triphosphate (8-Br-ATP),
8-Chloro-2′-deoxy-adenosine-5′-triphosphate (8-Cl-2′dATP),
8-Chloro-adenosine-5′-triphosphate (8-Cl-ATP),
8-Iodo-guanosine-5′-triphosphate (8-I-GTP),
8-Oxo-2′-deoxy-adenosine-5′-triphosphate (8-Oxo-2′dATP),
8-Oxo-2′-deoxy-guanosine-5′-triphosphate (8-Oxo-2′dGTP),
8-Oxo-adenosine-5′-triphosphate (8-Oxo-ATP),
8-Oxo-guanosine-5′-triphosphate (8-Oxo-GTP),
Adenine-2′-deoxy-arabinosyl-5′-triphosphate (Ara-2′dATP),
Adenine-2′-deoxy-xylosyl-5′-triphosphate (Xylo-2′dATP),
Adenine-2′-fluoro-2′-deoxy-arabinosyl-5′-triphosphate (2′F-2′d-Ara-ATP),
Adenine-2′-fluoro-2′-deoxy-arabinosyl-5′-triphosphate (2′F-2′d-Ara-ATP),
Adenine-arabinosyl-5′-triphosphate (Ara-ATP),
Adenine-xylosyl-5′-triphosphate (Xylo-ATP),
Biotin-16-7-deaza-7-aminoallyl-2′-dGTP (Biotin-16-7-Deaza-AA-dGTP)
Ganciclovir triphosphate (9-(1,2-Dihydroxy-2-propoxymethyl)guanine-5′-Triphosphate, GCV-TP),
Guanine-2′-deoxy-xylosyl-5′-triphosphate (Xylo-2′dGTP),
Guanine-xylosyl-5′-triphosphate (Xylo-GTP),
Guanine-2′-deoxy-arabinosyl-5′-triphosphate (Ara-2′dGTP),
Guanine-2′-fluoro-2′-deoxy-arabinosyl-5′-triphosphate (2′F-2′d-Ara-GTP),
Guanine-arabinosyl-5′-triphosphate (Ara-GTP),
Hypoxanthine-2′-deoxy-arabinosyl-5′-triphosphate (Ara-2′dITP),
Hypoxanthine-2′-deoxy-xylosyl-5′-triphosphate (Xylo-2′dITP),
Hypoxanthine-arabinosyl-5′-triphosphate (Ara-ITP),
Hypoxanthine-xylosyl-5′-triphosphate (Xylo-ITP),
Inosine-5′-triphosphate (ITP)
N1-Methyl-adenosine-5′-triphosphate (N1-Methyl-ATP),
N1-Methyl-guanosine-5′-triphosphate (N1-Methyl-GTP),
N²-Methyl-2-deoxy-guanosine-5′-triphosphate (N²-Methyl-dGTP),
N6-(4-Amino)butyl-2′-deoxy-adenosine-5′-triphosphate (N6-aminobutyl-2′dATP),
N6-(4-Amino)butyl-adenosine-5′-triphosphate (N6-aminobutyl-ATP),
N6-(6-Amino)hexyl-2′-deoxy-adenosine-5′-triphosphate (N6-aminohexyl-2′dATP),
N6-(6-Amino)hexyl-2′-deoxy-adenosine-5′-triphosphate-biotin (N6-aminohexyl-2′dATP-biotin),
N6-(6-Amino)hexyl-adenosine-5′-triphosphate (N6-aminohexyl-ATP),
N6-(6-Amino)hexyl-adenosine-5′-triphosphate-biotin (N6-aminohexyl-ATP-biotin),
N6-(6-Azido)hexyl-2′-deoxy-adenosine-5′-triphosphate (N6-azidohexyl-2′dATP),
N6-(6-Azido)hexyl-adenosine-5′-triphosphate (N6-azidohexyl-ATP),
N6-(6-Propargyl)-2′-deoxy-adenosine-5′-triphosphate (N6-propargyl-2′dATP),
N6-(6-Propargyl)-adenosine-5′-triphosphate (N6-propargyl-ATP),
N6-Benzyl-2′-deoxy-adenosine-5′-triphosphate (N6-benzyl-2′dATP),
N6-Benzyl-adenosine-5′-triphosphate (N6-benzyl-ATP),
N6-Methyl-2-deoxy-adenosine-5′-triphosphate (N6-Methyl-2′dATP),
N6-Methyl-adenosine-5′-triphosphate (N6-Me-ATP),
O6-Methyl-2′-deoxy-guanosine-5′-triphosphate (O6-Methyl-dGTP),
O6-Methyl-guanosine-5′-triphosphate (O6-Methyl-GTP),
Xanthine-2′-deoxy-arabinosyl-5′-triphosphate (2′d-Ara-XTP),
Xanthine-2′-deoxy-xylosyl-5′-triphosphate (2′d-Xylo-XTP),
Xanthine-arabinosyl-5′-triphosphate (Ara-XTP),
Xanthine-xylosyl-5′-triphosphate (Xylo-XTP) and
Xanthosine-5′-triphosphate (XTP).

29. A process for the preparation of cyclic di-guanosine monophosphate (cyclic-diGMP) by enzymatic synthesis comprising:
contacting the polypeptide of claim 1 with guanosine triphosphate (GTP) and isolating the cyclic-diGMP.

30. (canceled)

31. The process of claim 29, wherein the polypetide is immobilized, or immobilized to Ni-NTA resin.

32. (canceled)

33. The process of claim 29, wherein the polypeptide is contacted with its substrate at a temperature in the range from 0 to 100° C. and/or at a pH range from 5 to 12.

34-36. (canceled)

37. A method of identifying a polypeptide which has diguanylate cyclase activity and which has no phosphodiesterase activity, the method comprising:
(i) mutating one or more amino acids at positions 249 to 511 of the polypeptide comprising amino acids whose sequence is set forth as SEQ ID NO:1 to obtain a mutant,
(ii) determining the diguanylate cyclase activity of the mutant, and
(iii) determining the phosphodiesterase activity of the mutant,
wherein the presence of diguanylate cyclase activity and a absence of phosphodiesterase activity identifies the polypeptide which has diguanylate cyclase activity and which has no phosphodiesterase activity.

38-39. (canceled)


SEQUENCE LISTING (5 sequences)

SEQ ID NO: 1 - protein, 511 aa, Agrobacterium vitis
MVNQPFEVSEPDRIDRLTRRLERERAARRQAETLLEARARELYMANCQLARQAEDLEQRVMQRTEELVQERERAIALAQQDQLTGLANRRSFTAVLTDACQKSRLRGDRVTVVLVDMDRFKAINDAYGHEAGDAVLREFARRLMVAAGPGTVARLGGDEFALILDHLSDVTADHAFVSSFRASLQEPVLFNGKRLEVSASIGYASLPDHAASSTELLRHADMALYVSKKAGLSLATAFDDDLWAEANERRLLELELVLAMDRHEIMPWYQPIVDGASKRILGVEVLARWQHPYRGLVLPGVFLPLAEERNLLDRLFAQVARAACAQTAPLVKAGKLNYVSVNISPSQFKSGNLAMVIASILEQTGFPATALVVEITEDLIMSDLLRAGRELSELSRLGVRVALDDFGTGYSNISSLSSLPIQWLKLDRSLISRVGDDRVGQVIVQAVLQMAQALGIDVVAEGIETEVQARWLVEAGCRKHQGFLYGRPAPLEDLLCFSQPERLSLAAGKSP

SEQ ID NO: 2 - protein, 511 aa, Artificial Sequence
  AVL variant
MVNQPFEVSEPDRIDRLTRRLERERAARRQAETLLEARARELYMANCQLARQAEDLEQRVMQRTEELVQERERAIALAQQDQLTGLANRRSFTAVLTDACQKSRLRGDRVTVVLVDMDRFKAINDAYGHEAGDAVLREFARRLMVAAGPGTVARLGGDEFALILDHLSDVTADHAFVSSFRASLQEPVLFNGKRLEVSASIGYASLPDHAASSTELLRHADMALYVSKKAGLSLATAFDDDLWAEANERRLLELELVLAMDRHEIMPWYQPIVDGASKRILGVAVLARWQHPYRGLVLPGVFLPLAEERNLLDRLFAQVARAACAQTAPLVKAGKLNYVSVNISPSQFKSGNLAMVIASILEQTGFPATALVVEITEDLIMSDLLRAGRELSELSRLGVRVALDDFGTGYSNISSLSSLPIQWLKLDRSLISRVGDDRVGQVIVQAVLQMAQALGIDVVAEGIETEVQARWLVEAGCRKHQGFLYGRPAPLEDLLCFSQPERLSLAAGKSP

SEQ ID NO: 3 - protein, 511 aa, Artificial Sequence
  AAL variant
MVNQPFEVSEPDRIDRLTRRLERERAARRQAETLLEARARELYMANCQLARQAEDLEQRVMQRTEELVQERERAIALAQQDQLTGLANRRSFTAVLTDACQKSRLRGDRVTVVLVDMDRFKAINDAYGHEAGDAVLREFARRLMVAAGPGTVARLGGDEFALILDHLSDVTADHAFVSSFRASLQEPVLFNGKRLEVSASIGYASLPDHAASSTELLRHADMALYVSKKAGLSLATAFDDDLWAEANERRLLELELVLAMDRHEIMPWYQPIVDGASKRILGVAALARWQHPYRGLVLPGVFLPLAEERNLLDRLFAQVARAACAQTAPLVKAGKLNYVSVNISPSQFKSGNLAMVIASILEQTGFPATALVVEITEDLIMSDLLRAGRELSELSRLGVRVALDDFGTGYSNISSLSSLPIQWLKLDRSLISRVGDDRVGQVIVQAVLQMAQALGIDVVAEGIETEVQARWLVEAGCRKHQGFLYGRPAPLEDLLCFSQPERLSLAAGKSP

SEQ ID NO: 4 - DNA, 1536 bp, Artificial Sequence
  AVL variant
atggtaaaccagccatttgaggtttccgagcctgatcggatcgaccggctgacccgccgtctggagcgcgagcgcgcggcccgccgccaagccgagacattgctggaagcgcgtgcgcgtgaactttacatggccaattgccaattggcacgacaggcggaagatctcgaacagcgggtcatgcagcgcacggaggagctggttcaggagcgcgaacgtgccattgcactggcccagcaggaccaattgaccgggcttgccaatcgtcgcagctttaccgcggttttgaccgacgcttgccagaagagccgccttcgaggcgaccgtgttacggttgttcttgttgatatggataggttcaaggcgatcaacgacgcttatggtcatgaggcgggggatgcggtgctgcgggaatttgcccgccgcctgatggttgcggctggacctggaacggtggcacggctgggtggtgacgaatttgcattgattctcgaccatctgtcggatgtgacggccgatcatgcctttgtctcaagttttcgcgccagtttgcaggagccggttctcttcaacggcaagcgcctggaggtctcggcgtccatcggatatgcgagcctgccggatcatgccgcatcgtccaccgaattgctgcggcatgccgatatggcgctttacgtttccaagaaagctgggctgtcgctggcgaccgcttttgacgacgacctttgggccgaggccaacgagcgacgcttgctggagctggaactggtgctggccatggaccgccatgaaatcatgccctggtaccagccgatcgttgatggagccagcaagcggatactgggggtggccgtgctggcacgctggcagcatccctatcgcggtctggtgctgccgggtgtatttctgccactggcggaagagcgcaaccttctggatcggttgtttgcgcaggtcgcccgcgccgcctgtgcgcaaacagcgccgctggtcaaggctggcaagctcaactacgtgtcggtcaatatttctccaagccagttcaagtccggtaatctggcgatggtcatcgcgtccatcctggaacagaccggttttccagccacggcattggtggtggagattaccgaggacctgatcatgtcggacctgctgcgggccgggcgcgagctttccgaactttcgcggctcggcgttcgggttgcgctcgatgatttcggcaccggctattcgaatatttcctctctcagcagcctgccgatccaatggttgaaactagaccgtagcctgatcagccgggttggggacgaccgagttgggcaggttatcgttcaggccgtgctgcaaatggcccaggcgctcggcatcgatgttgtcgcggaaggtatcgaaacggaggtgcaggcacgctggcttgtggaagccggatgccgcaaacatcagggttttctctatggccgcccggcacctctggaagacttgctgtgtttctcgcaaccggaacgtctaagccttgcagctggaaagagcccttga

SEQ ID NO: 5 - DNA, 1536 bp, Artificial Sequence
  AAL variant
atggtaaaccagccatttgaggtttccgagcctgatcggatcgaccggctgacccgccgtctggagcgcgagcgcgcggcccgccgccaagccgagacattgctggaagcgcgtgcgcgtgaactttacatggccaattgccaattggcacgacaggcggaagatctcgaacagcgggtcatgcagcgcacggaggagctggttcaggagcgcgaacgtgccattgcactggcccagcaggaccaattgaccgggcttgccaatcgtcgcagctttaccgcggttttgaccgacgcttgccagaagagccgccttcgaggcgaccgtgttacggttgttcttgttgatatggataggttcaaggcgatcaacgacgcttatggtcatgaggcgggggatgcggtgctgcgggaatttgcccgccgcctgatggttgcggctggacctggaacggtggcacggctgggtggtgacgaatttgcattgattctcgaccatctgtcggatgtgacggccgatcatgcctttgtctcaagttttcgcgccagtttgcaggagccggttctcttcaacggcaagcgcctggaggtctcggcgtccatcggatatgcgagcctgccggatcatgccgcatcgtccaccgaattgctgcggcatgccgatatggcgctttacgtttccaagaaagctgggctgtcgctggcgaccgcttttgacgacgacctttgggccgaggccaacgagcgacgcttgctggagctggaactggtgctggccatggaccgccatgaaatcatgccctggtaccagccgatcgttgatggagccagcaagcggatactgggggtggccgccctggcacgctggcagcatccctatcgcggtctggtgctgccgggtgtatttctgccactggcggaagagcgcaaccttctggatcggttgtttgcgcaggtcgcccgcgccgcctgtgcgcaaacagcgccgctggtcaaggctggcaagctcaactacgtgtcggtcaatatttctccaagccagttcaagtccggtaatctggcgatggtcatcgcgtccatcctggaacagaccggttttccagccacggcattggtggtggagattaccgaggacctgatcatgtcggacctgctgcgggccgggcgcgagctttccgaactttcgcggctcggcgttcgggttgcgctcgatgatttcggcaccggctattcgaatatttcctctctcagcagcctgccgatccaatggttgaaactagaccgtagcctgatcagccgggttggggacgaccgagttgggcaggttatcgttcaggccgtgctgcaaatggcccaggcgctcggcatcgatgttgtcgcggaaggtatcgaaacggaggtgcaggcacgctggcttgtggaagccggatgccgcaaacatcagggttttctctatggccgcccggcacctctggaagacttgctgtgtttctcgcaaccggaacgtctaagccttgcagctggaaagagcccttga